(19) United States
(12) Patent Application Publication
(10) Pub. No.: US 2017/0238792 A1
(43) Pub. Date: Aug. 24, 2017

(54) OPTICAL FIBER SCANNER, ILLUMINATING DEVICE, AND OBSERVATION APPARATUS

(71) Applicant: OLYMPUS CORPORATION, Tokyo (JP)

(72) Inventors: Hirokazu YOKOTA, Tokyo (JP); Yasuaki KASAI, Saitama (JP); Hiroshi TSURUTA, Kanagawa (JP)

(73) Assignee: OLYMPUS CORPORATION, Tokyo (JP)

(21) Appl. No.: 15/588,931

(22) Filed: May 8, 2017

Related U.S. Application Data
(63) Continuation of application No. PCT/JP2014/079854, filed on Nov. 11, 2014.

(51) Int. Cl. A61B 1/07

(52) U.S. Cl. CPC A61B 1/07; A61B 1/00188

(57) ABSTRACT

Provided is an optical fiber scanner including: an illumination optical fiber that optically guides illumination light and outputs the light from a distal end thereof; an elastic section that engages with a base side of the illumination optical fiber relative to the distal end thereof and that is formed of an elastic material; piezoelectric elements fixed to a side surface of the elastic section, each piezoelectric element being polarized in a radial direction of the illumination optical fiber and receiving an alternating-current voltage in a polarization direction so as to cause the illumination optical fiber to vibrate via the elastic section; and a stationary section that has an engagement hole engaging with the elastic section at a position away from the piezoelectric elements toward the base side and that supports the illumination optical fiber in a cantilevered manner via the elastic section engaged with the engagement hole.

### CROSS-REFERENCE TO RELATED APPLICATIONS

This is a continuation of International Application PCT/JP2014/079854, with an international filing date of Nov. 11, 2014, which is hereby incorporated by reference herein in its entirety.

### TECHNICAL FIELD

The present invention relates to optical fiber scanners, illuminating devices, and observation apparatuses.

### BACKGROUND ART

A known optical fiber scanner in the related art outputs illumination light while vibrating the distal end of an optical fiber at high speed by using piezoelectric elements so as to scan the illumination light over a subject (for example, see Patent Literature 1). In the optical fiber scanner described in Patent Literature 1, the optical fiber is supported by an elastic section formed of a substantially prismatic member having a plurality of piezoelectric elements bonded thereto. The elastic section is joined to a ring-shaped support section, and the two sections are retained in an endoscope frame.

### CITATION LIST

### Patent Literature

{PTL 1}

Japanese Unexamined Patent Application, Publication No. 2013-244045

### SUMMARY OF INVENTION

The present invention provides the following solutions.

A first aspect of the present invention provides an optical fiber scanner including: an optical fiber that optically guides light and outputs the light from a distal end thereof; an elastic section that engages with a base side of the optical fiber relative to the distal end thereof and that is formed of an elastic material capable of transmitting vibrations to the optical fiber; a plurality of piezoelectric elements fixed to a side surface of the elastic section, each piezoelectric element being polarized in a radial direction of the optical fiber and receiving an alternating-current voltage in a polarization direction so as to cause the optical fiber to vibrate via the elastic section; and a support section that has an engagement hole engaging with the elastic section at a position away from the piezoelectric elements toward the base side and that is capable of supporting the optical fiber in a cantilevered manner via the elastic section engaged with the engagement hole. The support section and the elastic section have indicators that indicate joining positions in a state where the support section and the elastic section are engaged with each other.

A second aspect of the present invention provides an illuminating device including the aforementioned optical fiber scanner, a light source that generates light to be optically guided by the optical fiber, a focusing lens that focuses the light output from the optical fiber, and an outer casing that accommodates the focusing lens and the optical fiber scanner and that retains the support section.

A third aspect of the present invention provides an observation apparatus including the aforementioned illuminating device and a light detector that detects feedback light returning from a subject as a result of the subject being irradiated with light by the illuminating device.

### BRIEF DESCRIPTION OF DRAWINGS

FIG. 1 schematically illustrates the overall configuration of an endoscope apparatus according to an embodiment of the present invention.

FIG. 2 schematically illustrates the overall configuration of an optical fiber scanner in FIG. 1.

FIG. 3 is a cross-sectional view of an elastic section in FIG. 2, taken in the radial direction of an illumination optical fiber.

FIG. 4 is a cross-sectional view of a stationary section in FIG. 2, taken in the radial direction of the illumination optical fiber.

FIG. 5 schematically illustrates the overall configuration of an optical fiber scanner according to a first modification of the embodiment of the present invention.

FIG. 6 schematically illustrates the overall configuration of an optical fiber scanner according to a second modification of the embodiment of the present invention.

FIG. 7 illustrates an example of marks in the elastic section and the stationary section according to a third modification of the embodiment of the present invention.

FIG. 8 illustrates another example of marks in the elastic section and the stationary section according to the third modification of the embodiment of the present invention.

FIG. 9 illustrates the elastic section and the stationary section in FIG. 8, as viewed from the opposite direction.

### DESCRIPTION OF EMBODIMENTS

An optical fiber scanner, an illuminating device, and an observation apparatus according to an embodiment of the present invention will be described below with reference to the drawings.

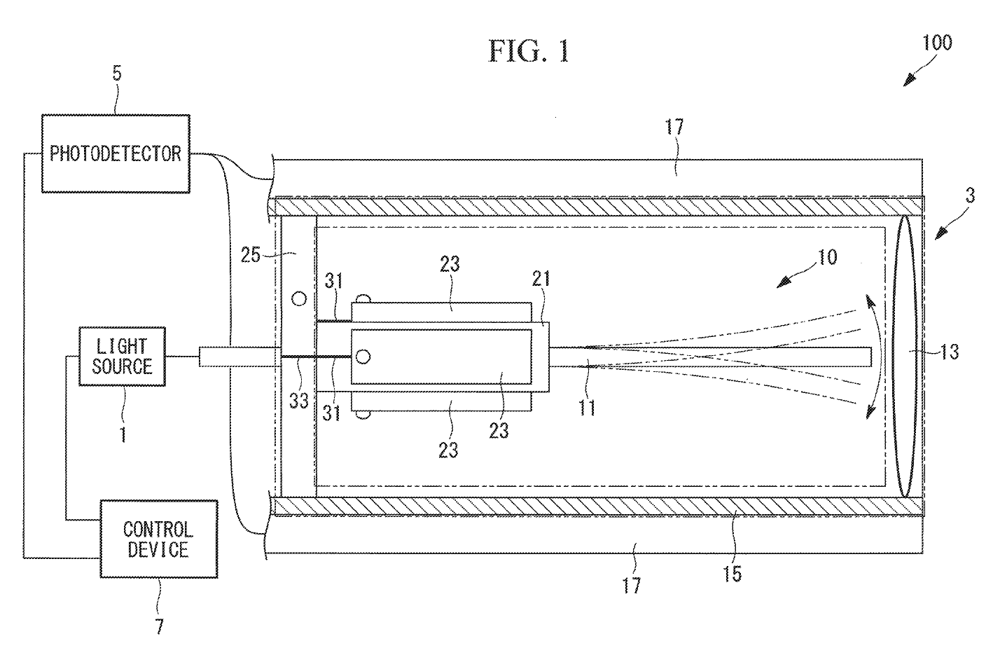

As shown in FIG. 1, an endoscope apparatus (observation apparatus) 100 according to this embodiment includes a light source 1 that generates illumination light, an illuminating device 3 that radiates the illumination light onto a subject (not shown), a photodetector (light detector) 5 that detects feedback light, such as reflected light or fluorescence, returning from the subject as a result of the subject being irradiated with the illumination light, and a control device 7 that controls, for example, the illuminating device 3 and the photodetector 5.

The illuminating device 3 includes an optical fiber scanner 10 having an illumination optical fiber 11 that optically guides the illumination light emitted from the light source 1 and outputs the illumination light from the distal end thereof, a focusing lens 13 that focuses the illumination light output from the illumination optical fiber 11, a narrow tubular endoscope frame (outer casing) 15 that accommodates the optical fiber scanner 10 and the focusing lens 13 therein, and a plurality of detection optical fibers 17 that are disposed on the outer peripheral surface of the endoscope frame 15 and that optically guide the feedback light from the subject to the photodetector 5.

The light source 1 and the photodetector 5 are disposed at the base end of the optical fiber scanner 10.

The control device 7 includes a CPU (not shown) that controls the illuminating device 3 and the photodetector 5, and also includes a memory that stores, for example, a program for actuating the CPU and various kinds of signals to be input to the CPU.

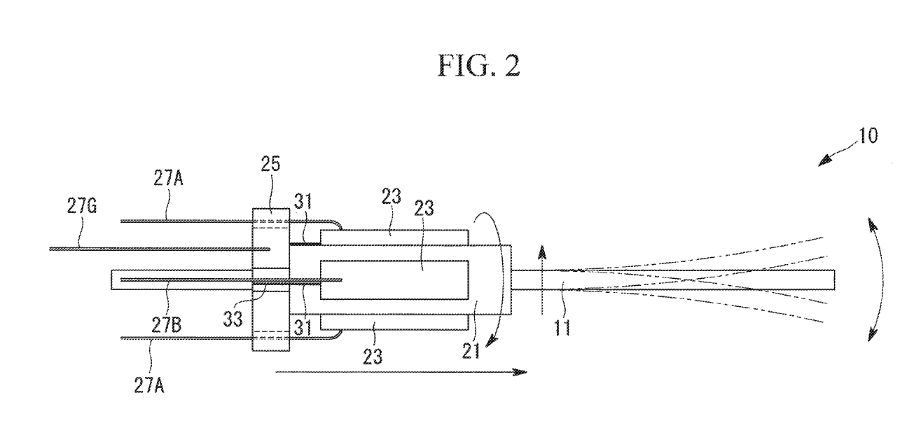

As shown in FIGS. 2 to 4, the optical fiber scanner 10 includes the illumination optical fiber 11 (optical fiber), such as a multi-mode fiber or a single-mode fiber, a tubular elastic section 21 that engages with the base side of the illumination optical fiber 11 relative to the distal end thereof and that is formed of an elastic material, four piezoelectric elements 23 fixed to the elastic section 21, a stationary section (support section) 25 that supports the illumination optical fiber 11 via the elastic section 21, and a driving lead wire (GND) 27G and four lead wires 27A and 27B.

As shown in FIGS. 1 and 2, the illumination optical fiber 11 is formed of a narrow glass material and is disposed in the longitudinal direction of the endoscope frame 15. With regard to the illumination optical fiber 11, one end thereof extends outward from the base end of the endoscope frame 15 so as to be connected to the light source 1, whereas the other end thereof is disposed near the distal end inside the endoscope frame 15.

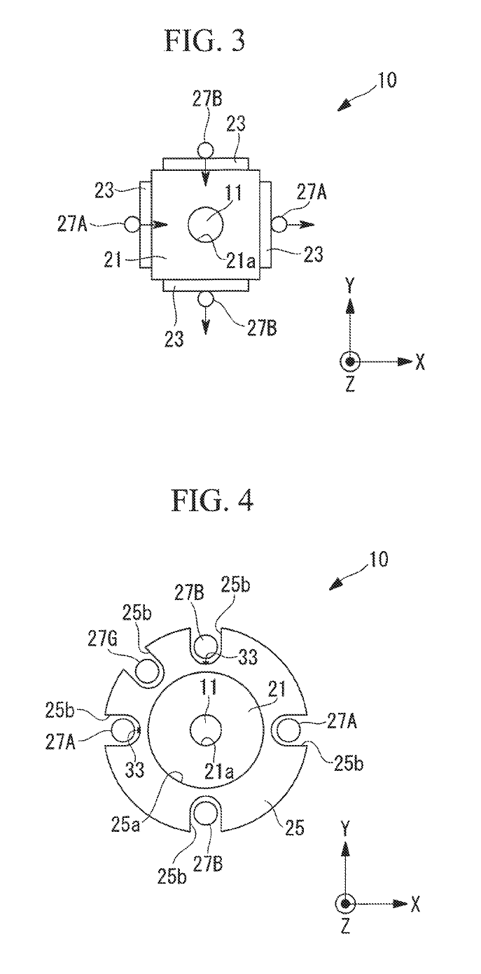

As shown in FIGS. 3 and 4, the elastic section 21 has an engagement hole 21a in which the illumination optical fiber 11 is fitted. In the engagement hole 21a, the engaged illumination optical fiber 11 is bonded thereto by using an epoxy-based adhesive applied to the outer peripheral surface of the illumination optical fiber 11.

Furthermore, as shown in FIG. 3, the elastic section 21 has a substantially quadratic prism shape and has the piezoelectric elements 23 respectively bonded to the four side surfaces thereof. The elastic section 21 transmits vibrations occurring in the piezoelectric elements 23 to the illumination optical fiber 11.

The stationary section 25 is ring-shaped, and the outer peripheral surface thereof is bonded to the inner wall of the endoscope frame 15 by using an epoxy-based adhesive. Moreover, as shown in FIG. 4, the stationary section 25 has an engagement hole 25a engageable with the elastic section 21.

The stationary section 25 engages with the elastic section 21 by means of the engagement hole 25a at a position away from the piezoelectric elements 23 toward the base end so as to support the illumination optical fiber 11 in a cantilevered manner. Thus, the stationary section 25 suppresses vibrations of the illumination optical fiber 11 occurring in the radial direction at this position. Supposing that the vibrations escape toward the base end of the illumination optical fiber 11 from the piezoelectric elements 23, the vibrations returning in a changed shape due to them receiving a certain effect can still be suppressed. Therefore, the stationary section 25 can prevent the vibrating shape of the piezoelectric elements 23 and the vibrations of the illumination optical fiber 11 from becoming unstable.

Furthermore, the stationary section 25 is electrically joined to electrodes on the back surfaces of the four piezoelectric elements 23 so as to function as a common ground (GND) when the piezoelectric elements 23 are driven. The stationary section 25 is joined to the lead wire 27G. Moreover, the stationary section 25 has, on the outer peripheral surface thereof, five grooves 25b that are recessed in the radial direction so as to be capable of accommodating the lead wire 27G and the four lead wires 27A and 27B therein.

The grooves 25b are circumferentially spaced apart from one another on the outer peripheral surface of the stationary section 25 and extend parallel to the central axis. Therefore, the lead wires 27A and 27B accommodated in the grooves 25b can be connected to the piezoelectric elements 23 without making them longer than necessary. For example, the four grooves 25b accommodating the four lead wires 27A and 27B are formed at positions equally spaced apart in the circumferential direction of the stationary section 25.

Furthermore, the five grooves 25b have a depth with which the lead wires 27A, 27B, and 27G can be substantially entirely accommodated therein so that an epoxy-based adhesive used for fixing the lead wires 27A, 27B, and 27G in the respective grooves 25b does not protrude therefrom. Consequently, the stationary section 25 can be accurately engaged with the endoscope frame 15.

The elastic section 21 and the stationary section 25 each have two linear marks (indicators) indicating the joining positions when they are engaged with each other. In FIG. 2, the marks in the elastic section 21 are each denoted by a reference sign 31. In FIGS. 2 and 4, the marks in the stationary section 25 are each denoted by a reference sign 33.

The two marks 31 in the elastic section 21 are positionally offset from each other by 90° in the circumferential direction on the outer peripheral surface at the base end of the elastic section 21 and extend parallel to the central axis.

The two marks 33 in the stationary section 25 extend parallel to the central axis at the bottom of two of the grooves 25b disposed at positions offset from each other by 90° in the circumferential direction of the stationary section 25.

In a state where the elastic section 21 is engaged with the stationary section 25, the two marks 31 in the elastic section 21 and the two marks 33 in the stationary section 25 are positioned so as to be linearly connected, when the elastic section 21 and the stationary section 25 are viewed in the radial direction, whereby the elastic section 21 and the stationary section 25 can be joined to each other in a positioned state around the central axis and in the radial direction.

The piezoelectric elements 23 are each composed of a piezoelectric ceramic material, such as lead zirconate titanate (PZT), and have a narrow plate-like shape. Furthermore, each piezoelectric element 23 is given a positive electrode treatment on the front surface thereof and a negative electrode treatment on the back surface thereof so as to be polarized from the positive electrode toward the negative electrode, that is, in the thickness direction.

As shown in FIG. 3, the four piezoelectric elements 23 are disposed on the respective side surfaces of the elastic section 21 at identical positions in the longitudinal direction of the illumination optical fiber 11. It is desirable that each piezoelectric element 23 and the stationary section 25 be separated from each other by at least a gap that prevents interference with expansion and contraction of the piezoelectric element 23 in a direction intersecting the polarization direction thereof. Accordingly, expansion and contraction of the piezoelectric element 23 in the longitudinal direction of the illumination optical fiber 11 are not interfered with by the stationary section 25.

Furthermore, as indicated by arrows denoting the directions of polarization in FIG. 3, each pair of piezoelectric elements 23 facing each other in the radial direction of the illumination optical fiber 11 is disposed such that the directions of polarization thereof are identical to each other. Moreover, by using a conductive epoxy-based adhesive, the lead wires 27A serving as A-phase lead wires are joined to the electrode surfaces of one of the pairs of piezoelectric elements 23, and the lead wires 27B serving as B-phase lead wires are joined to the electrode surfaces of the other pair of piezoelectric elements 23.

When an alternating-current voltage is applied in the polarization direction to the piezoelectric elements 23 via the lead wires 27A and 27B, vibrations (transverse effect) that cause the piezoelectric elements 23 to expand and contract in the direction orthogonal to the polarization direction occur. Moreover, the expansion and contraction occur in a manner such that when one of the piezoelectric elements 23 in each pair contracts, the other one expands concurrently therewith. Consequently, the piezoelectric elements 23 in each pair transmit the vibrations to the illumination optical fiber 11 via the elastic section 21 so as to cause the distal end of the illumination optical fiber 11 to vibrate in the direction intersecting the longitudinal direction.

The lead wire 27G is accommodated in one of the grooves 25b in the stationary section 25, and one end thereof is jointed to the groove 25b by using a conductive adhesive or by soldering. The lead wires 27A and 27B connected to the piezoelectric elements 23 are accommodated in the remaining grooves 25b in the stationary section 25 and are fixed to the grooves 25b by using an epoxy-based adhesive.

The detection optical fibers 17 are each formed of a narrow glass material and are disposed in the longitudinal direction on the outer peripheral surface of the endoscope frame 15. These detection optical fibers 17 are spaced apart from one another in the circumferential direction of the endoscope frame 15. With regard to each detection optical fiber 17, one end thereof is disposed at the distal end of the endoscope frame 15, whereas the other end thereof is connected to the photodetector 5.

In addition to controlling the illuminating device 3 and the photodetector 5, the control device 7 can generate image information by associating an intensity signal of feedback light detected by the photodetector 5 with information (scan-position information) related to the position of illumination light scanned by the optical fiber scanner 10.

The operation of the optical fiber scanner 10, the illuminating device 3, and the endoscope apparatus 100 having the above-described configuration will now be described.

In order to observe a subject by using the optical fiber scanner 10, the illuminating device 3, and the endoscope apparatus 100 according to this embodiment, the distal end of the endoscope frame 15 is first disposed facing the subject, and illumination light is generated from the light source 1. The illumination light emitted from the light source 1 is optically guided by the illumination optical fiber 11, is output from the distal end thereof, and is radiated onto the subject by the focusing lens 13.

When feedback light, such as reflected light or fluorescence, is generated in the subject as a result of the subject being irradiated with the illumination light, the feedback light is optically guided by the detection optical fibers 17 and is detected by the photodetector 5. Then, the control device 7 converts the feedback light into image information by associating an intensity signal of the feedback light output from the photodetector 5 with scan-position information of the optical fiber scanner 10. Consequently, an image of the subject irradiated with the illumination light can be generated.

Next, an illumination-light scanning process performed by the optical fiber scanner 10 will be described.

In order to cause the optical fiber scanner 10 to scan the illumination light, the bending resonance frequency of the illumination optical fiber 11 is first excited such that the central area of the stationary section 25 in the axial direction serves as a node and the distal end of the illumination optical fiber 11 serves as an antinode.

When an alternating-current voltage corresponding to the bending resonance frequency is applied to one of the pairs of piezoelectric elements 23 (referred to as “A-phase piezoelectric elements 23” hereinafter), the A-phase piezoelectric elements 23 vibrate. Then, the vibrations occurring in the A-phase piezoelectric elements 23 are transmitted to the illumination optical fiber 11 via the elastic section 21, thus causing the distal end of the illumination optical fiber 11 to vibrate in one direction (for example, the X-axis (A-phase) direction in FIGS. 3 and 4) intersecting the longitudinal direction.

Likewise, when an alternating-current voltage corresponding to the bending resonance frequency is applied to the other pair of piezoelectric elements 23 (referred to as “B-phase piezoelectric elements 23” hereinafter), the B-phase piezoelectric elements 23 vibrate. Then, the vibrations occurring in the B-phase piezoelectric elements 23 are transmitted to the illumination optical fiber 11 via the elastic section 21, thus causing the distal end of the illumination optical fiber 11 to vibrate in one direction (for example, the Y-axis (B-phase) direction in FIGS. 3 and 4) orthogonal to the X-axis direction.

By simultaneously causing the A-phase piezoelectric elements 23 to vibrate in the X-axis direction and the B-phase piezoelectric elements 23 to vibrate in the Y-axis direction and changing the phases of the alternating signals applied to the A-phase piezoelectric elements 23 and the B-phase piezoelectric elements 23 by n/2, the vibrations at the distal end of the illumination optical fiber 11 form a circular trajectory. By gradually adjusting (modulating) the magnitude of the alternating-current voltages applied to the A-phase piezoelectric elements 23 and the B-phase piezoelectric elements 23 in this state, the distal end of the illumination optical fiber 11 vibrates spirally. Consequently, the illumination light output from the distal end of the illumination optical fiber 11 can be scanned spirally over the subject.

In this case, during the manufacturing process of the optical fiber scanner 10 according to this embodiment, the elastic section 21 and the stationary section 25 are engaged with each other in a positioned state based on the marks 31 and 33 so that the joining position between the elastic section 21 and the stationary section 25 is fixed. This can prevent the joining of the elastic section 21 and the stationary section 25 from varying from optical fiber scanner 10 to optical fiber scanner 10. Consequently, the illumination optical fiber 11 and the focusing lens 13 can be readily and accurately positioned.

Furthermore, because the marks 31 in the elastic section 21 and the marks 35 in the stationary section 25 extend linearly in the longitudinal direction of the illumination optical fiber 11, the joining position in the radial direction and the circumferential direction of the illumination optical fiber 11 is clear. This allows for improved ease of positioning and improved joining accuracy between the elastic section 21 and the stationary section 25 in the radial direction and the circumferential direction of the illumination optical fiber 11.

Furthermore, in the illuminating device 3 according to this embodiment, the illumination optical fiber 11 and the focusing lens 13 are accurately positioned in accordance with the optical fiber scanner 10 having improved ease of assembly so that differences in performance among illuminating devices 3 can be suppressed. Therefore, the illumination light emitted from the light source 1 can be accurately scanned and radiated onto a desired position on the subject by the focusing lens 13. Moreover, with the endoscope apparatus 100 according to this embodiment, proper observation can be realized by acquiring image information of a desired observation region of the subject.

This embodiment can be modified as follows. In this embodiment, the elastic section 21 and the stationary section 25 respectively have the linear marks 31 and 33 as indicators extending parallel to the central axis. Alternatively, as a first modification, the elastic section 21 and the stationary section 25 may employ indicators extending orthogonally to the central axis.

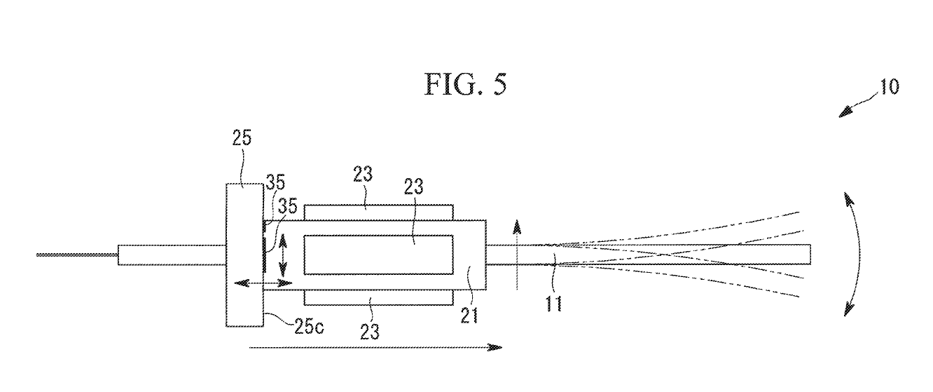

In this case, for example, as shown in FIG. 5, the elastic section 21 may have two linear marks 35 as indicators. Moreover, as an indicator, the stationary section 25 may employ a peripheral edge 25c of the end surface thereof located toward the distal end of the illumination optical fiber 11. Accordingly, the joining position in the longitudinal direction of the illumination optical fiber 11 is clear, so that the amount by which the stationary section 25 is pushed toward the elastic section 21 can be readily adjusted.

Therefore, in a state where the elastic section 21 is engaged with the stationary section 25, the two marks 35 in the elastic section 21 and the peripheral edge 25c of the end surface of the stationary section 25 are positioned so as to be linearly aligned, when the elastic section 21 and the stationary section 25 are viewed in the radial direction, whereby the elastic section 21 and the stationary section 25 can be joined to each other in a positioned state in the longitudinal direction of the illumination optical fiber 11. This allows for improved ease of positioning and improved joining accuracy between the elastic section 21 and the stationary section 25 in the longitudinal direction of the illumination optical fiber 11.

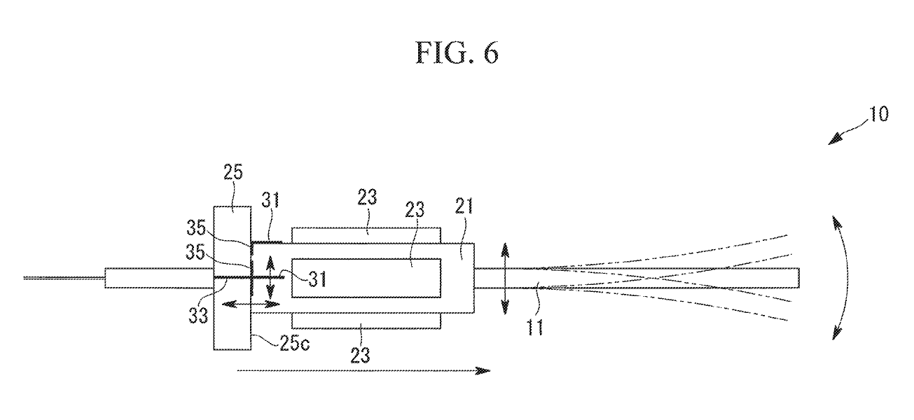

As a second modification, for example, as shown in FIG. 6, the elastic section 21 may have, as indicators, two linear marks 31 extending parallel to the central axis and two linear marks 35 extending orthogonally to the marks 31. Furthermore, the stationary section 25 may employ, as indicators, two linear marks 33 extending parallel to the central axis and the peripheral edge 25c of the end surface thereof located toward the distal end of the illumination optical fiber 11.

Accordingly, when the elastic section 21 and the stationary section 25 are viewed in the radial direction, the two marks 31 in the elastic section 21 and the two marks 33 in the stationary section 25 are positioned so as to be linearly connected, and the two marks 35 in the elastic section 21 and the peripheral edge 25c of the end surface of the stationary section 25 are positioned so as to be linearly aligned, whereby the elastic section 21 and the stationary section 25 can be joined to each other in a positioned state around the central axis, in the radial direction, and in the longitudinal direction.

As a third modification, for example, as shown in FIGS. 7 to 9, the elastic section 21 may be replaced with an elastic section 41 formed into a substantially prismatic shape by, for example, wire electrical discharge machining. Specifically, the substantially prismatic shape of the elastic section 41 is obtained by machining a cylindrical surface 41a of a columnar member having a diameter engageable with the engagement hole 25a of the stationary section 25 such that four sections of the cylindrical surface 41a, which are equally spaced in the circumferential direction, remain. Accordingly, the stationary section 25 and the elastic section 41 can be positioned in the radial direction by simply engaging the elastic section 41 with the stationary section 25.

In this case, as shown in FIG. 7, the elastic section 41 may have four linear marks (indicators) 43 that are provided in the end surface of the elastic section 41 at the opposite side from the distal end of the illumination optical fiber 11 and that extend toward the central axis from the respective cylindrical surfaces 41a. Moreover, the stationary section 25 may have four linear marks 45 provided in the end surface of the stationary section 25 at the opposite side from the distal end of the illumination optical fiber 11. The four marks 45 may be provided at positions equally spaced apart in the circumferential direction and may extend radially outward from the central axis.

Accordingly, in a state where the elastic section 41 is engaged with the stationary section 25, the four marks 43 in the elastic section 41 and the four marks 45 in the stationary section 25 are positioned so as to be linearly connected, when the stationary section 25 and the elastic section 41 are viewed in the axial direction, whereby the stationary section 25 and the elastic section 41 can be joined to each other in a positioned state in the circumferential direction in addition to the radial direction.

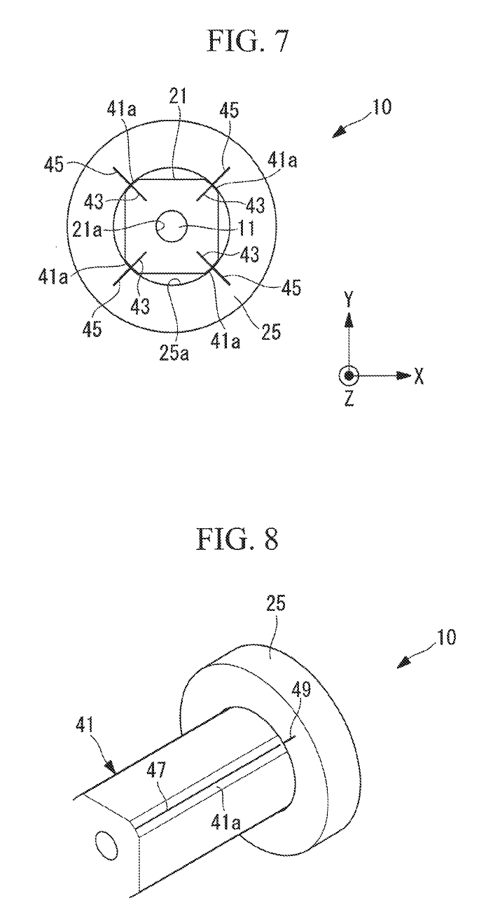

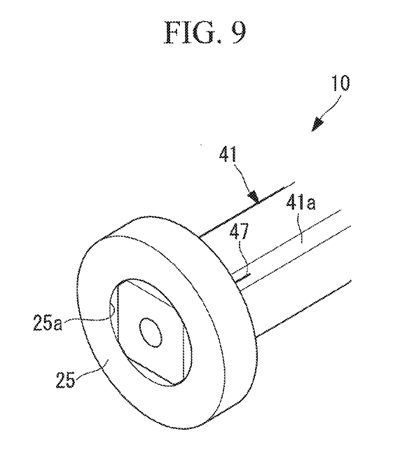

Furthermore, as shown in FIGS. 8 and 9, the cylindrical surfaces 41a of the elastic section 41 may individually have linear marks 47 that are provided at the end of the elastic section 41 at the opposite side from the distal end of the illumination optical fiber 11 and that extend parallel to the central axis. Moreover, the stationary section 25 may have four linear marks 49 provided in the end surface of the stationary section 25 located toward the distal end of the illumination optical fiber 11. The four marks 49 may be provided at positions equally spaced apart in the circumferential direction and may extend radially outward from the central axis.

In this case, in a state where the elastic section 41 is engaged with the stationary section 25, the four marks 47 in the elastic section 41 and the four marks 49 in the stationary section 25 are positioned so as to be linearly connected, when the elastic section 41 and the stationary section 25 are viewed in the central-axis direction, whereby the elastic section 41 and the stationary section 25 can be joined to each other in a positioned state in the circumferential direction and the longitudinal direction in addition to the radial direction.

Although the embodiment of the present invention has been described above in detail with reference to the drawings, specific configurations are not limited to this embodiment and include, for example, design alterations so long as they do not deviate from the scope of the invention. For example, the present invention is not limited to the above-described embodiment and the modifications thereof. The present invention is not particularly limited and may be applied to embodiments obtained by appropriately combining the embodiment and the modifications thereof.

Furthermore, in the embodiment and the modifications, the linear marks 31, 33, 35, 43, 45, 47, and 49 and the peripheral edge 25c of the end surface are employed as indicators. The indicators may be of any kind so long as they can indicate the joining positions between the elastic section 21 or 41 and the stationary section 25 in the engaged state, and may be, for example, arrows or color codes. Furthermore, the number of indicators is not limited to that described in the above embodiment and the modifications and may be any number with which the elastic section 21 or 41 and the stationary section 25 can be accurately joined to each other.

As a result, the above-described embodiment leads to the following aspects.

A first aspect of the present invention provides an optical fiber scanner including: an optical fiber that optically guides light and outputs the light from a distal end thereof; an elastic section that engages with a base side of the optical fiber relative to the distal end thereof and that is formed of an elastic material capable of transmitting vibrations to the optical fiber; a plurality of piezoelectric elements fixed to a side surface of the elastic section, each piezoelectric element being polarized in a radial direction of the optical fiber and receiving an alternating-current voltage in a polarization direction so as to cause the optical fiber to vibrate via the elastic section; and a support section that has an engagement hole engaging with the elastic section at a position away from the piezoelectric elements toward the base side and that is capable of supporting the optical fiber in a cantilevered manner via the elastic section engaged with the engagement hole. The support section and the elastic section have indicators that indicate joining positions in a state where the support section and the elastic section are engaged with each other.

According to this aspect, when an alternating-current voltage is applied to each piezoelectric element in the polarization direction thereof, the piezoelectric element vibrates by expanding and contracting in the direction orthogonal to the polarization direction, that is, the longitudinal direction of the optical fiber, and the vibrations of the piezoelectric element are transmitted to the optical fiber via the elastic section. Furthermore, the support section that supports the elastic section prevents the vibrations occurring in each piezoelectric element from escaping toward the base side of the optical fiber. Consequently, the distal end of the optical fiber is stably vibrated, so that the light output from the distal end of the optical fiber can be accurately scanned in accordance with the vibrations of the optical fiber.

During the manufacturing process of this optical fiber scanner, the elastic section and the support section are engaged with each other in a positioned state based on the indicators so that the joining position between the elastic section and the support section is fixed. This can prevent the joining of the elastic section and the support section from varying from optical fiber scanner to optical fiber scanner. Consequently, the optical fiber can be readily and accurately positioned relative to an illumination optical system that radiates the light output from the optical fiber onto a subject. The indicators may include, for example, marks added to the support section and the elastic section and an end surface of the support section or the elastic section.

In the above aspect, the indicator in at least one of the support section and the elastic section may be a mark added to an outer surface thereof.

With this configuration, desired positions of the support section and the elastic section can be set as the joining positions based on the marks when the two sections are engaged with each other, regardless of the shapes thereof.

In the above aspect, the indicators may each have a linear shape extending in a longitudinal direction of the optical fiber.

With this configuration, the joining position in the radial direction and the circumferential direction of the optical fiber is clear based on the indicators. This allows for improved ease of positioning and improved joining accuracy between the elastic section and the support section in the radial direction and the circumferential direction of the optical fiber.

In the above aspect, the indicators may each have a linear shape extending in a direction substantially orthogonal to a longitudinal direction of the optical fiber.

With this configuration, the joining position in the longitudinal direction of the optical fiber is clear based on the indicators. This allows for improved ease of positioning and improved joining accuracy between the elastic section and the support section in the longitudinal direction of the optical fiber.

A second aspect of the present invention provides an illuminating device including the aforementioned optical fiber scanner, a light source that generates light to be optically guided by the optical fiber, a focusing lens that focuses the light output from the optical fiber, and an outer casing that accommodates the focusing lens and the optical fiber scanner and that retains the support section.

According to this aspect, the optical fiber and the focusing lens are accurately positioned in accordance with the optical fiber scanner having improved ease of assembly, so that differences in performance among illuminating devices can be suppressed. Therefore, the light emitted from the light source can be accurately scanned and be radiated onto a desired position of a subject by the focusing lens.

A third aspect of the present invention provides an observation apparatus including the aforementioned illuminating device and a light detector that detects feedback light returning from a subject as a result of the subject being irradiated with light by the illuminating device.

According to this aspect, the light detector detects feedback light returning from a desired region of a subject over which light is scanned by the illuminating device. Therefore, proper observation can be realized by acquiring image information of a desired observation region of the subject.

The present invention is advantageous in that it can achieve improved ease of assembly by preventing variations in joining of an elastic section and a support section.

### REFERENCE SIGNS LIST

1 light source
3 illuminating device
5 photodetector (light detector)
10 optical fiber scanner
11 illumination optical fiber (optical fiber)
13 focusing lens
15 endoscope frame (outer casing)
21 elastic section
23 piezoelectric element
25 stationary section (support section)
31, 33, 35, 43, 45, 47, 49 mark (indicator)
100 endoscope apparatus (observation apparatus)

1. An optical fiber scanner comprising:
an optical fiber that optically guides light and outputs the light from a distal end thereof;
an elastic section that engages with a base side of the optical fiber relative to the distal end thereof and that is formed of an elastic material capable of transmitting vibrations to the optical fiber;
a plurality of piezoelectric elements fixed to a side surface of the elastic section, each piezoelectric element being polarized in a radial direction of the optical fiber and receiving an alternating-current voltage in a polarization direction so as to cause the optical fiber to vibrate via the elastic section; and
a support section that has an engagement hole engaging with the elastic section at a position away from the piezoelectric elements toward the base side and that is capable of supporting the optical fiber in a cantilevered manner via the elastic section engaged with the engagement hole,
wherein the support section and the elastic section have indicators that indicate joining positions in a state where the support section and the elastic section are engaged with each other.

2. The optical fiber scanner according to claim 1,
wherein the indicator in at least one of the support section and the elastic section is a mark added to an outer surface thereof.

3. The optical fiber scanner according to claim 1,
wherein the indicators each have a linear shape extending in a longitudinal direction of the optical fiber.

4. The optical fiber scanner according to claim 1,
wherein the indicators each have a linear shape extending in a direction substantially orthogonal to a longitudinal direction of the optical fiber.

5. An illuminating device comprising:
the optical fiber scanner according to claim 1;
a light source that generates light to be optically guided by the optical fiber;
a focusing lens that focuses the light output from the optical fiber; and
an outer casing that accommodates the focusing lens and the optical fiber scanner and that retains the support section.

6. An observation apparatus comprising:
the illuminating device according to claim 5; and
a light detector that detects feedback light returning from a subject as a result of the subject being irradiated with light by the illuminating device.
